(19) United States
(12) Patent Application Publication
(10) Pub. No.: US 2023/0363669 A1
(43) Pub. Date: Nov. 16, 2023

(54) BLOOD GLUCOSE METER ATTACHABLE TO DORSAL CARPAL ARTERY

(71) Applicant: TEMPUS INC., Seoul (KR)

(72) Inventor: Byoung Soo LEE, Namyangju-si Gyeonggi-do (KR)

(73) Assignee: TEMPUS INC., Seoul (KR)

(21) Appl. No.: 18/246,644

(22) PCT Filed: Mar. 23, 2021
(86) PCT No.: PCT/KR2021/003551

§ 371 Date: Mar. 24, 2023

(30) Foreign Application Priority Data
Sep. 24, 2020 (KR) 10-2020-0123626

(51) Int. Cl. A61B 5/145; A61B 5/1455; A61B 5/00

(52) U.S. Cl. CPC A61B 5/14532; A61B 5/1455; A61B 5/6824; A61B 5/0075

(57) ABSTRACT

The present invention relates to a blood glucose meter attachable to the wrist carotid artery, the blood glucose meter being attached near the carotid artery of the wrist, thereby being capable of greatly reducing delay time and enabling accurate and precise measurement of blood glucose. More particularly, the blood glucose meter attachable to the wrist carotid artery includes a body attached near carotid artery of wrist of human body and configured to block external light; a light emitting part installed in the body and configured to generate measurement light in a direction of the carotid artery; and a first light receiving part configured to receive reaction light to measure blood sugar from a photoreaction of blood flowing through the carotid artery to the measurement light.

### TECHNICAL FIELD

The present invention relates to a blood glucose meter attachable to the wrist carotid artery, and more particularly, to a blood glucose meter attachable to the wrist carotid artery, the blood glucose meter being attached near the carotid artery of the wrist, thereby being capable of greatly reducing delay time and enabling accurate and precise measurement of blood glucose.

### BACKGROUND ART

Invasive blood glucose meters collect blood from a user by means of a needle, a syringe, or the like and measure the blood glucose level. Accordingly, an invasive blood glucose meter causes physical pain to a user due to blood collection. In addition, if the invasive blood glucose meter is not maintained cleanly, a user may be infected with bacteria.

On the other hand, non-invasive blood glucose meters do not cause physical pain. As types of non-invasive blood glucose meters, there are a blood glucose meter using infrared rays, a blood glucose meter using an electromagnetic field, a blood glucose meter using exhalation, a blood glucose meter using a patch, and the like.

Among non-invasive blood glucose meters, a blood glucose meter using infrared rays measures the blood glucose level by irradiating the skin with infrared rays of various wavelengths and analyzing the reaction light of the skin to the infrared rays with a sensor. However, since the aspect of the reaction light is different depending on various situations such as skin conditions, physical characteristics, and distribution of blood vessels, it is difficult to accurately measure the blood glucose level due to a large deviation in the amount of light measured by a sensor.

In addition, a method of measuring the skin, earlobe, or inner lip has been proposed using such existing non-invasive blood glucose meters. However, a time delay may occur until sugar is digested and absorbed and the blood sugar in the blood spreads to an area to be measured.

For example, when measuring the inside of the lips, a time delay of about 5 minutes may occur until the blood glucose concentration of the body fluid at a measurement site inside the lips is reflected in the blood. Accordingly, there are problems in that rapid and precise measurement of blood sugar is difficult, so it is impossible to prepare for an emergency.

### DISCLOSURE

### Technical Problem

Therefore, the present invention has been made in view of the above problems, and it is one object of the present invention to provide a blood glucose meter attachable to the wrist carotid artery, the blood glucose meter being attached near the carotid artery of the wrist having a fast blood glucose reflection speed, thereby being capable of greatly reducing a delay time between the amount of blood sugar in the blood and the amount of blood glucose measured at a measurement site and enabling the rapid, accurate and precise measurement of blood sugar. It will be understood that the technical problems are only provided as examples, and the scope of the present invention is not limited thereto.

### Technical Solution

In accordance with an aspect of the present invention, the above and other objects can be accomplished by the provision of a blood glucose meter attachable to the wrist carotid artery, including: a body attached near carotid artery of wrist of human body and configured to block external light; a light emitting part installed in the body and configured to generate measurement light in a direction of the carotid artery; and a first light receiving part configured to receive reaction light to measure blood sugar from a photoreaction of blood flowing through the carotid artery to the measurement light.

In accordance with the present invention, an opening may be formed on one side of the body, a light transmission space is formed inside the opening to surround a vicinity of the carotid artery, and a reflector protrusion may be formed between the light emitting part and the first light receiving part to prevent the measurement light from being directly irradiated to the first light receiving part.

In accordance with the present invention, the first light receiving part may include a 1-1 light receiving part configured to detect light in a glucose emission (absorption) wavelength band of 9.4 μm to 9.8 μm to minimize influence of moisture and other body components; and a 1-2 light receiving part configured to detect light having a wavelength of 8.4 μm to 8.6 μm or 10.4 μm to 10.6 μm as a reference light.

In accordance with the present invention, the blood glucose meter may further include a second light receiving part for detecting light having a wavelength of 6.0 μm to 6.3 μm or 2.9 μm to 3.1 μm to measure skin moisture.

In accordance with the present invention, the blood glucose meter may further include a third light receiving part for detecting light of 5 μm to 14 μm to measure a temperature of skin.

In accordance with the present invention, the light emitting part may be a lamp capable of minimizing noise by using a differential signal for turning on and off a lamp.

In accordance with the present invention, the body may include an external light-blocking member for blocking external light; and an Attenuated Total Reflection (ATR) element installed inside the external light-blocking member, wherein a main light emitting line of the light emitting part is installed at one end of the ATR element to be inclined by a first angle with respect to a horizontal reference line, a main light receiving line of the first light receiving part is installed at another end of the ATR element to be inclined by a second angle with respect to the horizontal reference line, and the ATR element is formed lengthily along the horizontal reference line.

In accordance with the present invention, the ATR element may include a light guide formed to have a thin thickness compared to a length such that a portion of measurement light generated from the light emitting part is totally reflected in a zigzag shape and is received by the first light receiving part and a corresponding reaction light is collected after another part of the measurement light is delivered to the carotid artery direction of the wrist; a skin contact layer installed on a front of the light guide and made of a skin-friendly translucent material; and a reflective layer installed on a rear surface of the light guide and configured to reflect the measurement light that is not totally reflected.

In accordance with the present invention the light guide may be a crystal including at least one of Ge, Si, ZnSe, and ZnS and combinations thereof, and the skin contact layer may include a High-Density PolyEthylene (HDPE) component.

### Advantageous Effects

In accordance with some embodiments of the present invention configured as described above, a non-invasive blood glucose meter is attached near the carotid artery of the wrist with a fast blood glucose reflection speed, thereby being capable of greatly reducing a delay time and enabling accurate and precise measurement of blood glucose. It is natural that the scope of the present invention is not limited by the effects.

### DESCRIPTION OF DRAWINGS

FIG. 1 is a use state diagram illustrating a blood glucose meter attachable to the wrist carotid artery according to some embodiments of the present invention.

FIG. 2 is a sectional view conceptionally illustrating the blood glucose meter attachable to the wrist carotid artery of FIG. 1.

FIG. 3 is a graph illustrating an example of a light absorption spectrum applied to the blood glucose meter attachable to the wrist carotid artery of FIG. 1.

FIG. 4 is a graph illustrating another example of a light absorption spectrum applied to the blood glucose meter attachable to the wrist carotid artery of FIG. 1.

FIG. 5 is a sectional view conceptionally illustrating a blood glucose meter attachable to the wrist carotid artery according to some other embodiments of the present invention.

FIG. 6 is a perspective view illustrating the blood glucose meter attachable to the wrist carotid artery of FIG. 5.

### BEST MODE

Hereinafter, one or more preferred embodiments of the present invention will be described in detail with reference to the accompanying drawings.

Embodiments of the present invention are provided to more completely explain the present invention to those skilled in the art, and the following embodiments may be modified in many different forms, but the scope of the present invention is not limited to the following embodiments. Rather, the embodiments are provided to make the invention thorough and complete and to fully convey the technical idea of the invention to those skilled in the art. In the drawings, the thicknesses and sizes of layers may be exaggerated for convenience and clarity of explanation.

Hereinafter, a blood glucose meter according to various embodiments of the present invention is described in detail with reference to the accompanying drawings.

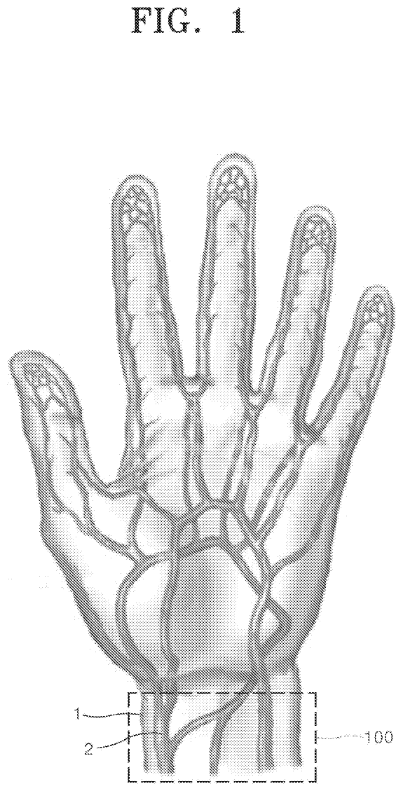

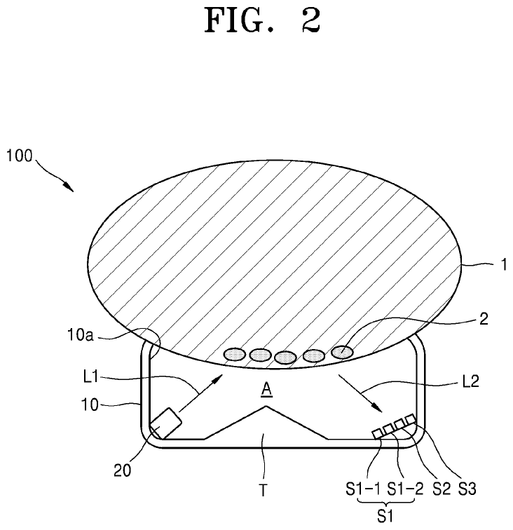

FIG. 1 is a use state diagram illustrating a blood glucose meter 100 attachable to the wrist carotid artery according to some embodiments of the present invention, and FIG. 2 is a sectional view conceptionally illustrating the blood glucose meter 100 attachable to the wrist carotid artery of FIG. 1.

First, as shown in FIGS. 1 and 2, the blood glucose meter 100 attachable to the wrist carotid artery according to some embodiments of the present invention may be attached or worn near the carotid artery of the wrist 1 of the human body and may largely include a body 10, a light emitting part 20 and a first light receiving part S1.

For example, as shown in FIGS. 1 and 2, the body 10 may be attached near the carotid artery 2 of the wrist 1 of the human body or may be worn like a smart band or a smart watch, may have a box-shaped structure with one side open capable of blocking external light, may be made of a metal-plated synthetic resin or a metal material, and may have various block or frame structures having sufficient strength and durability to support the light emitting part 20 and the first light receiving part S1 described above. However, the shape of the body 10 is not limited to the shape shown in the drawing, and very various types of structures in which a space can be formed may be applied.

As a more specific example, an opening 10a formed on one side of the body 10, a light transmission space A is formed inside the opening 10a to surround the vicinity of the carotid artery 2, and a reflector protrusion T may be formed between the light emitting part 20 and the first light receiving part S1 to prevent measurement light L1 generated from the light emitting part 20 from being directly irradiated to the first light receiving part S1.

Accordingly, the measurement light L1 may be guided in a direction of the carotid artery 2 by the reflector protrusion T, and only the reaction light L2 of the carotid artery 2 for this measurement light L1 may be received by the first light receiving part S1.

Here, the reflector protrusion T forms a kind of blocking wall, and a tip of the reflector protrusion T is not necessarily limited to the drawing and may be formed in a wide variety of shapes such as being pointed in a triangular shape or formed in a round shape.

For example, as shown in FIGS. 1 and 2, the light emitting part 20 may be installed on the body 10 and may include an infrared emitting body such as an infrared LED or infrared lamp that generates the measurement light L1 in the direction of the carotid artery 2. For example, an infrared light emitting device having a wavelength of 2.5 μm to 3.5 μm having the strongest water absorption at a center wavelength may be applied to the light emitting part 20. The emitted infrared light in the range of 2.5 μm to 3.5 μm is absorbed by moisture of the skin, and the tissues whose temperature is elevated by the infrared absorption emit characteristic radiation corresponding to an increased temperature. The characteristic radiation rays generated at this time coincide with the characteristic absorption line of tissue in a low-temperature state according to Kirchhoff's law. Therefore, the components of the tissue may be inferred by measuring the radiation rays.

As a more specific example, the light emitting part 20 may be a lamp capable of minimizing noise by using a differential signal for turning on and off a lamp.

For example, as shown in FIGS. 1 and 2, the first light receiving part S1 may be a kind of light receiving device, such as a sensor, an optical sensor or a light receiving element, installed inside the body 10 and configured to receive the reaction light L2 so as to measure blood sugar from photoreaction of the blood flowing through the carotid artery with respect to the measurement light L1.

The first light receiving part S1 uses the characteristics of light emission (absorption) spectrum which is described below and may selectively receive the light of a wavelength of a specific band by using a bandpass filter or a pattern or material of an element.

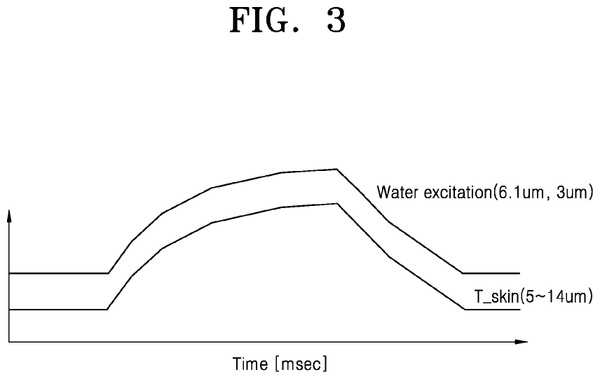

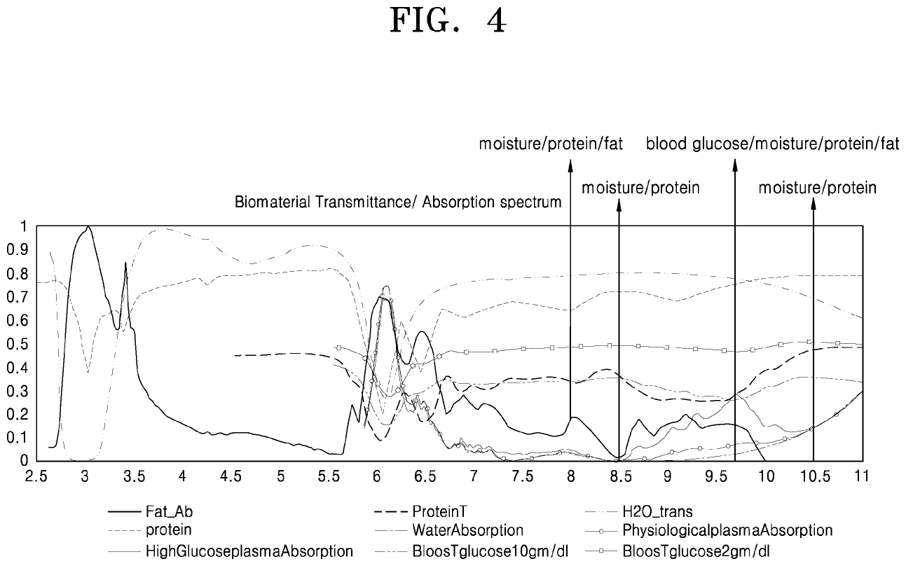

FIG. 3 is a graph illustrating an example of a light emission (absorption) spectrum applied to the blood glucose meter 100 attachable to the wrist carotid artery of FIG. 1, and FIG. 4 is a graph illustrating another example of a light emission (absorption) spectrum applied to the blood glucose meter 100 attachable to the wrist carotid artery of FIG. 1.

As shown in FIGS. 1 to 4, light receiving parts of the present invention which are described below may discriminate a material by using a valley portion, i.e., a light emission (absorption) characteristic of a specific band, of a graph having a high light emission (absorption) rate in a light emission (absorption) spectrum.

As a more specific example, as shown in FIGS. 1 to 4, the first light receiving part S1 may include a 1-1 light receiving part S1-1 that detects light in a glucose absorption wavelength band of 9.4 μm to 9.8 μm to minimize influence of moisture and other body components (salt, protein, fat, etc.); and a 1-2 light receiving part S1-2 that detects light having a wavelength of 8.4 μm to 8.6 μm or 10.4 μm to 10.6 μm as a reference light.

Here, for example, a bandpass filter having a center wavelength of 10.27 μm and a bandwidth of 210 nm or a center wavelength of 8.3 μm and a bandwidth of 250 nm may be applied to the 1-2 light receiving part S1-2.

In addition, the blood glucose meter 100 attachable to the wrist carotid artery according to some embodiments of the present invention may further include a second light receiving part S2 for detecting light having a wavelength of 6.0 μm to 6.3 μm or 2.9 μm to 3.1 μm to measure skin moisture, as shown in FIGS. 1 to 4.

Here, the second light receiving part S2 is also a kind of light receiving device, such as a sensor, an optical sensor or a light receiving element, installed inside the body 10 and may selectively receive light having a wavelength of 6.0 μm to 6.3 μm or 2.9 μm to 3.1 μm using a bandpass filter or a pattern or material of an element by using the characteristics of a light emission (absorption) spectrum.

For example, a bandpass filter having a central wavelength of 6.23 μm and a bandwidth of 200 nm may be applied to the second light receiving part S2.

Accordingly, skin moisture may be measured using the second light receiving part S2 of the blood glucose meter 100 attachable to the wrist carotid artery of the present invention, and a blood glucose level compared with a reference light measured in the 1-2 light receiving part S1-2 may be very accurately calculated by detecting light in a glucose light emission (absorption) wavelength band using the 1-1 light receiving part S1-1 based on the total amount of the moisture.

Therefore, a delay time may be greatly reduced and the blood glucose may be rapidly and accurately measured by attaching near the carotid artery of the wrist with a fast blood glucose reflection speed in consideration of skin moisture and reference light according to the present invention.

For example, as shown in FIGS. 1 to 4, the blood glucose meter 100 attachable to the wrist carotid artery according to some embodiments of the present invention may further include a third light receiving part S3 for detecting light of 5 μm to 14 μm to measure the temperature of the skin.

Here, the third light receiving part S3 is also a kind of light receiving device such as a sensor, an optical sensor or a light receiving element, installed in the body 10 and may selectively receive light having a wavelength of 5 μm to 14 μm using a bandpass filter or a pattern or material of an element by using the characteristics of a light emission (absorption) spectrum.

Accordingly, the temperature of the skin may be measured using the third light receiving part S3 of the blood glucose meter 100 attachable to the wrist carotid artery according to the present invention, and a final blood glucose level may be very accurately calculated by correcting a blood glucose level based on the skin temperature.

Therefore, a delay time may be greatly reduced and the blood glucose may be rapidly and accurately measured by attaching near the carotid artery of the wrist with a fast blood glucose reflection speed in consideration of skin temperature according to the present invention.

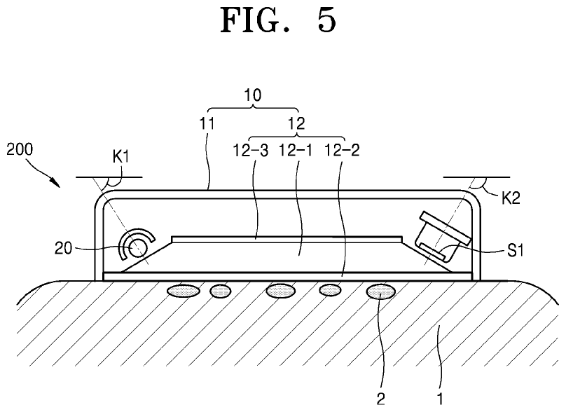

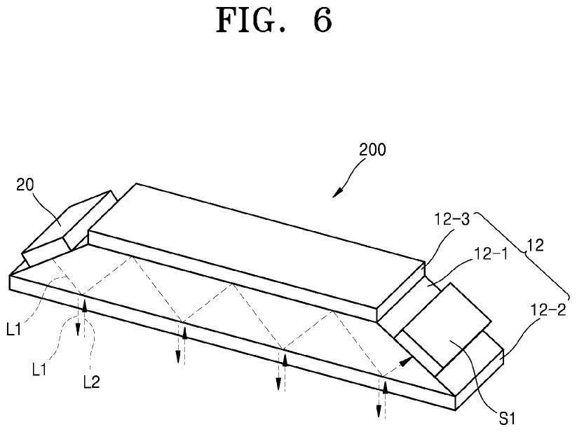

FIG. 5 is a sectional view conceptionally illustrating a blood glucose meter attachable to the wrist carotid artery 200 according to some other embodiments of the present invention, and FIG. 6 is a perspective view illustrating the blood glucose meter 200 attachable to the wrist carotid artery of FIG. 5.

As shown in FIGS. 5 and 6, a body 10 of the blood glucose meter 200 attachable to the wrist carotid artery according to some other embodiments of the present invention may include an external light-blocking member 11 for blocking external light; and an Attenuated Total Reflection (ATR) element 12 installed inside the external light-blocking member 11. A main light emitting line of the light emitting part 20 is installed at one end of the ATR element 12 to be inclined by a first angle K1 with respect to the horizontal reference line, a main light receiving line of the first light receiving part S1 is installed at another end of the ATR element 12 to be inclined by a second angle K2 with respect to the horizontal reference line, and the ATR element 12 is formed lengthily along the horizontal reference line.

The overlapping absorption and reflection by ATR crystal are mainly used for analysis of a material (e.g. liquid) on a surface in contact with the ATR crystal. When this is applied to the carotid artery in contact with the ATR crystal, the process “light originating from the light emitting part 20 is absorbed and reflected by the carotid artery 2, and the reflected light is reflected from the reflective surface of the ATR and enters the carotid artery 2 again” is repeated, so the radiation and absorption signals of the carotid artery 2 are amplified. That is, a signal that greatly changes may be obtained even with a small change in the amount of blood sugar and water.

Accordingly, more reaction light (L1) may be received through a wide sensing surface of the ATR element 12 by attaching the ATR element 12 near the carotid artery 2 of the wrist 1 of the human body.

As a more specific example, as shown in FIGS. 5 and 6, the ATR element 12 may include a light guide 12-1 that has a thin thickness compared to the length such that a portion of measurement light generated from the light emitting part 20 may be totally reflected in a zigzag shape and may be received by the first light receiving part S1 and the reaction light L2 can be collected after another part of the measurement light L1 is delivered to the carotid artery direction of the wrist; a skin contact layer 12-2 installed on the front of the light guide 12-1 and made of a skin-friendly translucent material; and a reflective layer 12-3 installed on a rear surface of the light guide 12-1 and configured to reflect the measurement light L1 that is not totally reflected.

Here, the light guide 12-1 may be, for example, a crystal including at least one of Ge, Si, ZnSe, and ZnS, which are materials having a high total reflectivity, and combinations thereof.

In addition, the skin contact layer 12-2 may include, for example, a High-Density PolyEthylene (HDPE) component that has high light transmittance, is skin-friendly, and does not have a large difference in refractive index with the skin compared to ATR crystal.

On the other hand, although not shown, various pressure sensors, heart rate sensors, oxygen saturation sensors, etc. may be additionally installed in the ATR element 12 or the skin contact layer 12-2 to measure blood pressure, heart rate, oxygen saturation, etc. as well as blood sugar of the user.

Accordingly, as shown in FIG. 6, the measurement light L1 generated from the light emitting part 20 may be totally reflected in a zigzag form inside the light guide 12-1, and in the process, a portion of the measurement light L1 may be irradiated over a wide area in the direction of the carotid artery 2 of the wrist 1 of the human body. The corresponding reaction light L2 may also be collected over a large area and received by the first light receiving part S1 through total reflection.

Therefore, light may be irradiated and collected over a wider area, and blood glucose may be accurately and precisely measured by using the optical characteristics of the collected light, i.e., the light emission (absorption) spectrum of the carotid artery 2 of the wrist 1 of the human body. In addition, blood pressure, heart rate, oxygen saturation, and the like in addition to blood sugar may be measured.

Although the present invention has been described with reference to embodiments shown in the drawings, the embodiments are provided as only exemplary examples, and those skilled in the art will understand that various modifications and equivalent other embodiments are possible therefrom. Therefore, the true technical protection scope of the present invention should be determined by the technical spirit of the appended claims.

### INDUSTRIAL APPLICABILITY

A non-invasive blood glucose meter according to some embodiments of the present invention configured as described above is attached near the carotid artery of the wrist with a fast blood glucose reflection speed without causing physical pain, thereby being capable of greatly reducing greatly reduce a delay time between the amount of blood sugar in the blood and the amount of blood glucose measured at a measurement site. This enables rapid, accurate and precise measurement of blood sugar, so that a user can quickly prepare for emergencies.

1. A blood glucose meter attachable to wrist carotid artery, comprising:
a body attached near carotid artery of wrist of human body and configured to block external light;
a light emitting part installed in the body and configured to generate measurement light in a direction of the carotid artery; and
a first light receiving part configured to receive reaction light to measure blood sugar from a photoreaction of blood flowing through the carotid artery to the measurement light.

2. The blood glucose meter according to claim 1, wherein an opening is formed on one side of the body, a light transmission space is formed inside the opening to surround a vicinity of the carotid artery, and a reflector protrusion is formed between the light emitting part and the first light receiving part to prevent the measurement light from being directly irradiated to the first light receiving part.

3. The blood glucose meter according to claim 2, wherein the first light receiving part comprises:
a 1-1 light receiving part configured to detect light in a glucose emission (absorption) wavelength band of 9.4 μm to 9.8 μm to minimize influence of moisture and other body components; and
a 1-2 light receiving part configured to detect light having a wavelength of 8.4 μm to 8.6 μm or 10.4 μm to 10.6 μm as a reference light.

4. The blood glucose meter according to claim 1, further comprising a second light receiving part for detecting light having a wavelength of 6.0 μm to 6.3 μm or 2.9 μm to 3.1 μm to measure skin moisture.

5. The blood glucose meter according to claim 1, further comprising a third light receiving part for detecting light of 5 μm to 14 μm to measure a temperature of skin.

6. The blood glucose meter according to claim 1, wherein the light emitting part is a lamp capable of minimizing noise by using a differential signal for turning on and off a lamp.

7. The blood glucose meter according to claim 1, wherein the body comprises:
an external light-blocking member for blocking external light; and
an Attenuated Total Reflection (ATR) element installed inside the external light-blocking member,
wherein a main light emitting line of the light emitting part is installed at one end of the ATR element to be inclined by a first angle with respect to a horizontal reference line, a main light receiving line of the first light receiving part is installed at another end of the ATR element to be inclined by a second angle with respect to the horizontal reference line, and the ATR element is formed lengthily along the horizontal reference line.

8. The blood glucose meter according to claim 7, wherein the ATR element comprises:
a light guide formed to have a thin thickness compared to a length such that a portion of measurement light generated from the light emitting part is totally reflected in a zigzag shape and is received by the first light receiving part and a corresponding reaction light is collected after another part of the measurement light is delivered to the carotid artery direction of the wrist;
a skin contact layer installed on a front of the light guide and made of a skin-friendly translucent material; and
a reflective layer installed on a rear surface of the light guide and configured to reflect the measurement light that is not totally reflected.

9. The blood glucose meter according to claim 8, wherein the light guide is a crystal comprising at least one of Ge, Si, ZnSe, and ZnS and combinations thereof, and
the skin contact layer comprises a High-Density PolyEthylene (HDPE) component.
